(19) United States
(12) Patent Application Publication
(10) Pub. No.: US 2019/0021995 A1
(43) Pub. Date: Jan. 24, 2019

(54) FORMULATION FOR TREATMENT OF PERIPHERAL JOINTS, SPINAL JOINTS AND/OR EXTRACELLULAR MATRIX ELEMENTS OF CONNECTIVE TISSUE, METHOD OF MANUFACTURE AND USES

(71) Applicants: Alexander Vilenovich ASAFOV, Moscow (RU); Livio ORSINI GRAVINA, Singapore (SG)

(72) Inventors: Alexander Vilenovich ASAFOV, Moscow (RU); Livio ORSINI GRAVINA, Singapore (SG)

(73) Assignees: Alexander Vilenovich ASAFOV, Moscow (RU); Livio ORSINI GRAVINA, Singapore (SG)

(21) Appl. No.: 16/067,705

(22) PCT Filed: Dec. 30, 2015
(86) PCT No.: PCT/RU2015/000964

§ 371 Date: Jul. 2, 2018

(51) Int. Cl. A61K 9/06; A61K 47/10; A61K 47/14; A61K 47/34; A61K 31/7008; A61K 47/18; A61K 47/38; A61K 47/20; A61K 47/22; A61K 47/08; A61K 47/02; A61K 47/46; A61K 47/44; A61K 9/00; A61K 9/107; A61K 9/127; A61P 19/02

(52) U.S. Cl. CPC A61K 9/06; A61P 19/02; A61K 47/14; A61K 47/34; A61K 31/7008; A61K 47/18; A61K 47/38; A61K 47/20; A61K 47/22; A61K 47/08; A61K 47/02; A61K 47/46; A61K 47/44; A61K 9/0014; A61K 9/1075; A61K 9/127; A61K 47/10

(57) ABSTRACT

The invention belongs to the field of pharmaceutics and relates to formulations for external use for the treatment of peripheral joints, spinal joints and/or extracellular matrix elements of connective tissue. The claimed formulation provides for superior (in comparison to existing analogs) transdermal delivery of pharmaceutically active ingredients, in particular, glucosamine sulfate or other glucosamine salts, and additional ingredients to affected joints. Also proposed is a method of producing said formulation and therapeutic use thereof. The efficacy of the proposed formulation is confirmed by clinical test results.

### FIELD OF THE INVENTION

The invention belongs to the field of pharmaceutics. More specifically, it relates to topical formulations for the treatment of diseases of peripheral joints, spinal joints and/or extracellular matrix elements of connective tissue.

### BACKGROUND OF THE INVENTION

Glucosamine (2-amino-2-deoxy-D-glucose) and, in particular, glucosamine sulfate potassium chloride, is a specific substrate and stimulator of the synthesis of endogenous glycosaminoglycans. It belongs to the class of amino saccharides and is a universal precursor and building block of glycosaminoglycans and, further, proteoglycans, which, in turn, are constituents of the extracellular matrix of connective tissue of an organism (fascia, ligaments, sinews, cartilages, joints, articular capsule, and intervertebral disks).

Glucosamine promotes restoration of cartilaginous surfaces of peripheral joints and spinal joints, including intervertebral disks, and prevents erosion thereof. It also restores bone tissue, which erodes from under cartilage during late stages of joint diseases, increases elasticity and resilience of cartilaginous tissue and its resistance to mechanical stress, increases the amplitude of movements in joints, improves the quality of synovial fluid and improves microcirculation, improves tropism of articular tissue and decreases edema.

Also, glucosamine possesses anti-inflammatory and analgesic properties. It reduces the need for nonsteroidal anti-inflammatory drugs (NSAID), allows for reducing NSAID doses or eliminating NSAID treatment altogether. It suppresses formation of superoxide radicals and production of enzymes damaging cartilaginous tissue (collagenases and phosphol ipases).

Glucosamine has proven to be an effective chondroprotector in prevention and treatment of bone and connective tissue metabolism disorders, in particular, in collagen and cartilage matrix, which causes inflammatory and degenerative changes [1]. The concentration of endogenous glucosamine, which is a specific substrate and structural unit of glycosaminoglycans and proteoglycans in synovial fluid has a great effect on development and course of degenerative diseases. In case of a lack of endogenous glucosamine, glucosamine formulations replenish glucosamine in blood plasma and synovial fluid, thereby slowing down and even reversing cartilage erosion.

Glucosamine formulations are widely represented by oral, injection and external dosage forms containing glucosamine hydrochloride or glucosamine sulfate, intended for administration as a single dose ranging from 100 to 1500 mg.

Glucosamine-based formulations often include additional ingredients dimethyl sulfide (DMS; its other name is methylsulfonylmethane (MSM)) and ascorbic acid, which have positive therapeutic effects on the organism. More specifically, DMS has analgesic and anti-inflammatory effects. In addition, it promotes inactivation of hydroxyl radicals, improvement in metabolic processes at the site of inflammation and slows nociceptive impulses in peripheral neurons. DMS antagonizes vascular effects of histamine, bradykinin and prostaglandin E1.

Ascorbic acid has pronounced antioxidant and antiplatelet properties, suppresses production of prostaglandins and other mediators of inflammation and allergic reactions, and is involved in regulation of oxidation-reduction processes, carbohydrate metabolism, blood coagulation, and tissue regeneration.

Prior art discloses an agent for treatment of joint diseases that comprises a glucosamine salt, DMS, ointment base and non-steroidal anti-inflammatory drug (patent RU 2259204 C1, issued Aug. 27, 2005; patent EA 007597 B1, issued Dec. 29, 2006).

Prior art discloses an agent for treatment of joint diseases that comprises a glucosamine salt, a chondroitin sulfate salt and DMS included in an acceptable ointment base (patent RU 2271812 C1 issued Mar. 20, 2006).

Prior art discloses an agent for the care for peripheral joints and the spine that comprises a mixture of chondroitin sulfate, glucosamine sulfate, acetyl glucosamine and several variants of ointment bases (patent RU 2376011 issued Dec. 20, 2009).

Prior art discloses a pharmaceutical composition for treatment of joint diseases, comprising chondroitin sulfate, glucosamine salt, MSM, troxerutin, N-methyl-2-pyrollidone and ointment base (patent RU 2408380 issued Jan. 10, 2011).

Prior art discloses a composition for treatment of joint and muscle pain, consisting of capsaicinoid, glucosamine or salt thereof and ointment base (patent U.S. Pat. No. 6,689,399 issued Feb. 10, 2004).

Prior art discloses a composition for treating rheumatoid arthritis comprising a therapeutically effective amount of chondroitin sulfate, N-acetyl D-glucosamine and hyaluronan in the absence of an analgesic agent (patent application US20080003258 published Jan. 3, 2008).

Prior art discloses a transdermal joint pain therapy composition, comprising 0.1 to 15% glucosamine sulfate sodium chloride and a transdermal composition consisting of one or more substances selected from the group: cetyl alcohol, stearyl alcohol, stearic acid, glyceryl monostearate, isopropyl myristate, lecithin, butylated hydroxyl toluene, simethicone, urea, potassium sorbate, sodium hydroxide, polyoxyl 40 stearate, EDTA disodium, and water (patent application US20080102107 published May 1, 2008).

However, all existing glucosamine-based formulations have drawbacks which reduce the benefit from using them in practice. The reason that limits the use of oral glucosamine formulations in practice is the need to use high glucosamine concentrations and long-term oral administration to provide for necessary bioavailability, which may result in substantial irritation of gastrointestinal tract and adverse side effects associated therewith.

The existing external dosage forms (cream, ointment, emulsion, gel) comprising chondroitin and glucosamine do not have an effective mechanism of delivering active and excipient ingredients through skin and tissues to the synovial membrane surrounding the affected area. Therefore, despite the potential high efficacy of chondroitin and glucosamine, the therapeutic effect of classical topical agents is much less than the oral form.

Glucosamine formulations for injection, which provide maximal bioavailability, are limited by the very method of administration, which is not acceptable for all patients, as well as by the need for having trained health care professionals administer them.

All the above-listed formulations for transdermal delivery lack a physically structured transdermal carrier for the pharmaceutically active agents, in particular, for glucosamine and salts thereof. The passing of the above-mentioned ingredients (which have molecular weights between 179.2 (glucosamine) and 456.4 (glucosamine sulfate)) through skin results in delivery of fairly insignificant amounts of the active agent. Introduction into formulations excipients that improve the permeability of skin, such as, for example, DMS, only slightly increases the bioavailability of the above-mentioned pharmaceutically active agents. It should also be noted that DMS, which is most frequently declared as an ingredient promoting increased in skin permeability, may have adverse effects, which leads to a number of contraindications. For example, DMS is contraindicated in cases of severe cardiovascular insufficiency, atherosclerosis, kidney or liver disorders, stroke, pregnancy, comas, angina, breastfeeding, glaucoma and cataract. DMS can increase the adverse effects of other medicaments. In accordance with restrictions, DMS is to be used with caution by children below the age of 9 and seniors.

Patent application US20050232980 (published Oct. 20, 2005) discloses a composition in the form of a suspension for transdermal delivery of glucosamine and chondroitin sulfate comprising 0.01% to 20% of glucosamine; 0.01% to 20% of chondroitin sulfate; 0.01% to 10% of camphor; 0.01% of menthol; 0.01% to 20% of an anti-inflammatory agent; and a transdermal carrier in the form of lecithin organogel, MSM, benzyl alcohol, benzoic acid, or combinations thereof (ready-to-use lecithin organogel, alone or mixed with other chemical ingredients mentioned, is admixed with pharmaceutically active and auxiliary components mentioned above). Micelles of said lecithin organogel have a proven ability to easily penetrate skin and, in theory, they may positively affect the bioavailability of the pharmaceutically active ingredient in question. However, lecithin organogels exist in three-component systems comprising lecithin, a non-polar organic solvent, and water in a very narrow range of concentrations of water corresponding to tenths and hundredths of a percent by weight [3]. Therefore, the ability of lecithin organogel to transport pharmaceutically significant amounts of water-soluble ingredients, such as glucosamine and its salts, is very limited.

Thus, there is need for topical agents providing more efficient transdermal delivery of pharmaceutically active and auxiliary ingredients to affected joint area.

### SUMMARY OF THE INVENTION

In an formulation for treatment of peripheral joints, spinal joints and/or extracellular matrix elements of connective tissue proposed in accordance with the present invention a fundamentally new method of transdermal delivery of pharmaceutically active and auxiliary ingredients to tissues is used—namely, a system of transdermal delivery based on micellar and/or liposomal structures, which comprises an aggregative and sedimentation stable disperse system comprising at least the following ingredients:

60% to 80% by weight of water,

1.0% to 6.0% by weight of glycerol,

0.7% to 4% by weight of butylene glycol,

up to 12.1% by weight of C8 fatty alchohols,

up to 3.5% by weight of C16 fatty alchohols,

up to 4.2% by weight of C21 fatty alchohols,

up to 2.0% by weight of esters of C15-C17 fatty acids,

up to 1.4% by weight of polysiloxane,

up to 1.4% by weight of C2 polyglycol,

up to 0.98% by weight of C20 polyglycol.

Superior transdermal delivery is provided by a disperse system consisting of at least one or two groups of structures differing in hydrodynamic diameter/size, wherein the first group consists of micellar nanocapsules between 0.1 and 100 nm in size comprising active and auxiliary ingredients surrounded by/associated with a lipid layer, and a second group comprising micellar and/or liposomal structures 200 to 8000 nm in size.

The proposed delivery system provides for glucosamine delivery to target cells with efficiency comparable to that provided by intramuscular injection and several times higher than that provided by tablets or conventional ointments. In addition, in terms of anesthetic efficiency, formulations according to the invention act at the same level as, or even better than, commercially available non-steroidal anti-inflammatory drugs (NSAID). In terms of anesthetic efficiency and restoration of mobility of affected joint, as well as reduction or elimination of needed doses of NSAID, transdermal formulations according to the invention correspond, or are even superior, to known glucosamine-based agents for injection.

Topical application of an active agent according to the invention prevents degradation of the active components by gastrointestinal tract enzymes and provides a significantly greater therapeutic effect.

The proposed formulation combines two principles for combatting osteoarthritis: quick anti-inflammatory and anesthetic effect; and stable restoration of affected tissues and strengthening of cartilage characterized by a prolonged post-treatment effect. In addition, the proposed formulation for treatment of peripheral joints, spinal joints and/or extracellular matrix elements of connective tissue is safe during long-term use and has few if any side effects.

The technical result of the present invention is increase the pharmacological efficacy of topical agents for treatment of peripheral joints, spinal joints and/or extracellular matrix elements of connective tissue by significantly increasing the bioavailability of pharmaceutically active ingredients and auxiliary ingredients in blood plasma and synovial fluid surrounding the affected site.

The proposed drug formulation has substantially more pronounced therapeutic and anesthetic effects compared to existing formulations, due to more efficient transdermal delivery of pharmaceutically active and excipients to the affected joint, in particular, transdermal delivery of glucosamine sulfate and other glucosamine salts.

### BRIEF DESCRIPTION OF THE FIGURES

FIG. 1. Distribution of the number (relative volume) of micelles/liposomes by size in a sample of Formulation A.

FIG. 2. Glucosamine concentration (μg/mL) versus time (h) profile in rat plasma after applying Formulation A on shaved skin of the animal.

FIG. 3. Total WOMAC score (VAS) over time during treatment in 2 groups.

FIG. 4. Total WOMAC functional insufficiency score (VAS) over time during treatment in 2 groups.

FIG. 5. Average weekly NSAID (ibuprofen) requirement during treatment in 2 (two) groups.

### DETAILED DESCRIPTION OF THE INVENTION

The expression “% by weight” used throughout the specification to indicate the amount of an ingredient means percent ratio of the weight of said ingredient in the proposed formulation to the total weight of said formulation.

In accordance with the present invention, the formulation for the treatment of peripheral joints, spinal joints and/or extracellular matrix elements of connective tissue utilizes a fundamentally new method of transdermal delivery of polar pharmaceutically active ingredients, in particular, glucosamine, and excipients, such as DMS and/or ascorbic acid, to the micro-capillary blood circulation of the dermis and tissues surrounding the affected organ. More specifically, in addition to one or more pharmaceutically active ingredients, the disclosed formulation comprises a system for transdermal delivery that is based on micellar and liposomal structures and comprises an aggregative and sedimentation stable disperse system comprising at least the following ingredients:

60% to 80% by weight of water,

1.0% to 6.0% by weight of glycerol,

0.7% to 4% by weight of butylene glycol,

up to 12.1% by weight of C8 fatty alcohols,

up to 3.5% by weight of C16 fatty alcohols,

up to 4.2% by weight of C21 fatty alcohols,

up to 2.0% by weight of esters of C15-C17 fatty acids,

up to 1.4% by weight of polysiloxane,

up to 1.4% by weight of C2 polyglycol,

up to 0.98% by weight of C20 polyglycol.

The micellar and liposomal structures dispersed within the formulation may form at least two groups differing in hydrodynamic diameter/size, wherein the distribution of the number (relative volume) of structures versus their characteristic size resembles the Gaussian curve with a maximum.

More specifically, the first group of said structures consists of micellar nanocapsules 0.1-100 nm in size, in particular, 15-80 nm, wherein the hydrodynamic diameter/size of at least 90% of the structures is between 15 and 100 nm, and the maximum of the distribution of the number (relative volume) of particles is between 25 and 50 nm; each micelle is surrounded by artificial lipid coating similar to keratinocyte membranes and includes/associated with active and excipients.

The second group consists of micellar and/or liposomal structures 200 to 8000 nm in size, in particular, 500 to 7000 nm, wherein the hydrodynamic diameter/size of at least 90% of the structures may be between 500 and 7000 nm, and the maximum of the distribution of the number (relative volume) of particles may be between 1500 and 5000 nm; the structures of the group also include/associated with active and excipients.

Owing to their specific composition and size, nanocapsules of the first group of structures are capable of travelling easily in the intercellular space, without deforming or damaging tissue cells. When the formulation is massaged into the skin, the micelles pass in an efficient and noninjurious manner through the pores and intercellular space of dermal cells and the microcapillary bed vessels into blood stream. The artificial biological membrane of the micelles degrades upon contacting blood plasma and releases glucosamine and excipients, which diffuse from blood stream into the synovial fluid of the affected joint or first into tissues surrounding the affected joint and then into the affected joint itself.

As the formulation is massaged into the skin, the larger structures belonging the second group pass in an efficient and noninjurious manner partly through intercellular space, but mainly through the pores and derma channels, and release glucosamine and auxiliary ingredients into microcapillary bed vessels, which diffuse into blood stream and then into the synovial fluid of the affected joint or first into tissues surrounding the affected joint and then into the affected joint itself.

As studies aimed at determining an optimal makeup of the disclosed formulations have shown, it is the above-indicated list of constituents in the relative amounts indicated that form the micellar and liposomal structures that provide their high penetration capacity and aggregative and sedimentation stability, which, in turn, provide for superior bioavailability of pharmaceutically active ingredients, and storage stability of the drug for a time period of at least three years.

In addition, it is the indicated makeup of the disperse system that provides the structures, in particular, micells and/or liposomes, via implementation of the method of manufacture of the claimed formulation described here. Also, only the amounts of ingredients contained in the disperse system that are indicated in the description of the formulations and the method of manufacture provide for the aggregative and sedimentation stability of the proposed formulation in combination with its high penetration capacity, provided the production technology described herein is followed.

In accordance with the present invention, the above-indicated studies have shown that the transdermal delivery system described above can be used for delivering, for example, any glucosamine salt such as glucosamine hydrochloride, glucosamine sulfate, glucosamine sulfate stabilized with sodium chloride or potassium chloride. However, it is the use of glucosamine sulfate potassium chloride that has been found to provide maximal bioavailability of glucosamine in blood plasma and synovial fluid.

In one embodiment of the invention, the proposed formulation is used for the treatment of peripheral joints, spinal joints and/or extracellular matrix elements of connective tissue.

In one embodiment, said “one or more pharmaceutically active ingredients” is a glucosamine salt, such as glucosamine sulfate potassium chloride, wherein the glucosamine salt, in particular, glucosamine sulfate potassium chloride, is present in the formulation in the amount of 4% to 14% by weight. In oneembodiment, said amount ranges from 7.0% to 9.5% by weight. In another embodiment, said amount is 8.00% by weight.

In another embodiment, the one or more pharmaceutically active ingredients are present in the proposed formulation in an effective amount. In some embodiments of the invention the disclosed formulation may further comprise other pharmaceutically active ingredients, for example, heparin, ketoprofen, lidocaine and other compounds.

In addition to one or more pharmaceutically active ingredients, the proposed formulation comprises at least the following additional ingredients:

60% to 80% by weight of water,

1.0% to 6.0% by weight of glycerol,

0.7% to 4% by weight of butylene glycol,

up to 12.1% by weight of C8 fatty alcohols,

up to 3.5% by weight of C16 fatty alcohols,

up to 4.2% by weight of C21 fatty alcohols,

up to 2.0% by weight of esters of C15-C17 fatty acids,

up to 1.4% by weight of polysiloxane,

up to 1.4% by weight of C2 polyglycol,

up to 0.98% by weight of C20 polyglycol.

In one embodiment, water is present in the proposed formulation in the amount of 67-75% by weight; in one embodiment water is present in the amount of 71.69% by weight.

In another embodiment, glycerol is present in the formulation in the amount of 2.0-4.0% by weight; in one embodiment, glycerol is present in the amount of 3.0% by weight.

In another embodiment, butylene glycol is present in the formulation in the amount of 1.0-3.0% by weight; in one embodiment, butylene glycol is present in the amount of 2.0% by weight.

In one embodiment, the component C8 fatty alcohols are caprylic/capric triglycerides. In another embodiment, the C8 fatty alcohols, for example, caprylic/capric triglycerides, are present in the formulation in the amount of 1.7-12.1% by weight, in another embodiment, in the amount of 3.0-7.0% by weight and, in yet another embodiment, in the amount of 5.0% by weight.

In one embodiment, the C16 fatty alcohols are represented by cetyl alcohol. In another embodiment the C16 fatty alcohols, for example, cetyl alcohol, are present in the formulation in the amount of 0.3-3.5% by weight; in another embodiment, in the amount of 1.5-3.0% by weight and, in yet another embodiment, in the amount of 2.3% by weight.

In one embodiment of the invention, the C21 fatty alcohols are represented by glyceryl stearate. In another embodiment the C21 fatty alcohols, for example, glyceryl stearate, are present in the formulation in the amount of 1.0-4.2% by weight; in another embodiment, in the amount of 3.0-4.0% by weight and, in yet another embodiment, in the amount of 3.8% by weight.

In one embodiment, esters of C15-C17 fatty acids are represented by cetyl octanoate. In another embodiment, esters of C15-C17 fatty acids, for example, cetyl octanoate, are present in the formulation in the amount of 0.3-2.0% by weight, in another embodiment, in the amount of 0.7-1.3% by weight and, in yet another embodiment, in the amount of 1.0% by weight.

In another embodiment, polysiloxane is present in the formulation in the amount of 0.3-1.4% by weight; in another embodiment, in the amount of 0.3-1.0% by weight. In other embodiments, polysiloxane is not present in the formulation.

In another embodiment, the C2 polyglycol is steareth-2. In another embodiment the C2 polyglycol, for example, steareth-2, is present in the formulation in the amount of 0.3-1.4% by weight; in another embodiment, in the amount of 0.7-1.3% by weight and, in yet another embodiment, in the amount of 1.0% by weight.

In one embodiment of the invention, the C20 polyglycol is steareth-20. In another embodiment the C20 polyglycol, for example, steareth-20, is present in the formulation in the amount of 0.22-0.98% by weight; in another embodiment in the amount of 0.50-0.80% by weight and, in yet another embodiment, in the amount of 0.66% by weight.

In one embodiment, one or more additional ingredients comprise amino polycarboxylic acid in the amount of 0.05-0.3% by weight (this acid belongs to a family of ligands and acts as sequestrant regulating stability of a product), hydroxyethyl cellulose in the amount of 0.12-1.0% by weight and/or silicagel in the amount of 0.005-0.05% by weight, which act as rheological modifiers that allow for regulating the viscosity of a product.

In another embodiment, one or more additional ingredients comprise DMS in the amount of 0.5-5% by weight.

In another embodiment, one or more additional ingredients comprise ascorbic acid in the amount of 0.05-0.3% by weight.

In another embodiment, one or more additional ingredients comprise preservatives and/or perfume.

In another embodiment, one or more additional ingredients comprise capsaicinoid, in particular, capsaicin, in the amount of 0.05-0.5% by weight, camphor in the amount of 0.2-1.8% by weight, ginger extract in the amount of 0.00005-0.005% by weight, peppermint oil and/or menthol in the amount of 0.02-1.5% by weight, which, when applied to the skin, have a “warming” or partly anesthetic effect thereby imparting comfort to the patient. In particular, said peppermint oil and/or menthol in the amount of 0.02-1.5% by weight can be used in the formulation as perfume.

In another embodiment, one or more additional ingredients comprise standard preservatives, in particular, methylparaben in the amount of 0.01-0.3%, in one embodiment, in the amount of 0.2% by weight and/or methylisothiazolinone in the amount of 0.01-0.07%, and in one embodiment, in the amount of 0.05% by weight. To prepare a “preservative-free” formulation, a mixture of salix alba bark extract with propanediol (ISCAGUARD® SAP) in the amount of 0.02-2.5% by weight may be used to replace conventional preservatives.

In another embodiment, one or more additional ingredients comprise a buffer solution, in particular, Tris(hydroxymethyl)aminomethane (HOCH₂)3CNH₂ (hereinafter “Tris”) in the amount of 0.1-0.9% by weight.

In another embodiment one additional ingredients comprises silicagel in the amount of 0.005-0.05% by weight, which may serve as an ingredient of the micellar and liposomal structures formed in the disperse system.

In one embodiment, the proposed formulation has the following makeup

| | |
|---|---|
| water | 71.69% by weight |
| glycerol | 3.0% by weight |
| butylene glycol | 2.0% by weight |
| cetyl alcohol | 2.3% by weight |
| glyceryl stearate | 3.8% by weight |
| caprylic/capric triglycerides | 5.0% by weight |
| cetyl octanoate | 1.0% by weight |
| dimethicone | 1.0% by weight |
| steareth-2 | 1.0% by weight |
| steareth-20 | 0.66% by weight |
| glucosamine sulfate potassium chloride | 8.00% by weight |
| methylisothiazolinone | 0.05% by weight |
| methylparaben | 0.2% by weight |
| peppermint oil | 0.3% by weight. |

To prepare the proposed formulation for treatment of peripheral joints, spinal joints and/or extracellular matrix elements of connective tissue, a method of production is used that comprises the following steps:

(i) high-purity water in the amount of 60-80% by weight is added to a first mixing tank, followed by addition of glycerol in the amount of 1-6% by weight and butylene glycol in the amount of 0.7-4% by weight; the mixture is heated to 65-70° C. with constant stirring;

(ii) one or more C16 fatty alcohols in the amount of 0.3-3.5% by weight, one or more C21 fatty alcohols in the amount of 1-4.2% by weight, one or more C8 fatty alcohols in the amount of 1.7-12.1% by weight, one or more esters of C15-C17 fatty acids in the amount of 0.3-2.0% by weight, one or more polysiloxanes in the amount of 0.3-1.4% by weight, one or more polyglycols C2 in the amount of 0.3-1.4% by weight, and one or more polyglycols C20 in the amount of 0.22-0.98% by weight are added to a second mixing tank; the mixture is heated to 65-70° C. with constant stirring;

(iii) the mixture of (ii) is added to the first mixing tank with constant stirring during 5 minutes, followed by cooling the mixture down to 50° C. or less and adding, with constant stirring, one or more pharmaceutically active agents, followed by cooling the mixture down to 30° C. and adding, with constant stirring, preservative(s) and perfume, thereby producing a formulation for the treatment of peripheral joints, spinal joints and/or extracellular matrix elements of connective tissue.

In said method, the one or more pharmaceutically active agents comprise a glucosamine salt, in particular, glucosamine sulfate or glucosamine sulfate potassium chloride.

Also in said method, amino polycarboxylic acid in the amount of 0.05-0.3% and/or hydroxyethyl cellulose in the amount of 0.12-1.0% by weight and/or silicagel in the amount of 0.005-0.05% by weight may further be added at step (i) to the first mixing tank before the heating, to produce effects mentioned above in the description of said ingredients.

Also in one embodiment, in step (iii) of said method the addition of one or more pharmaceutically active agents is followed by adding further DMS in the amount of 0.5-5% by weight and/or ascorbic acid in the amount of 0.05-0.3% by weight, to produce effects mentioned above in the description of said ingredients.

Also in another embodiment, in step (ii) of said method the heating of the mixture in the second mixing tank to 65-70° C. is followed by adding further a capsaicinoid, in particular, capsaicin, in the amount of 0.05-0.5% by weight, to produce effects mentioned above in the description of said ingredients.

In another embodiment, in step (iii) of said method the addition of one or more pharmaceutically active ingredients is followed by adding camphor in the amount of 0.2-1.8% by weight and/or ginger extract in the amount of 0.00005-0.005% by weight, to produce effects mentioned above in the description of said ingredients.

In another embodiment, in step (iii) of said method methylparaben in the amount of 0.01-0.3% by weight and/or methylisotiazolinone in the amount of 0.01-0.05% by weight is added as preservative(s), or a mixture of salix alba bark extract with propanediol (ISCAGUARD® SAP) in the amount of 0.02-2.5% by weight is added as replacement of standard preservatives.

Also some embodiments, in step (iii) of said method the addition of one or more pharmaceutically active agents and cooling down to 30° C. is followed by adding peppermint oil and/or menthol in the amount of 0.02-1.5% by weight, both of which are perfume and impart comfort to a patient.

In step (i) of some embodiments of said method, Tris (hydroxymethyl) aminomethane in the amount of 0.1-0.9% by weight is added to the first mixing tank as a buffer solution after adding water, in order to maintain the pH of the formulation during long-term storage.

In one embodiment, a method of treatment is disclosed, in which the formulation containing an active agent is applied onto the skin at the site of the affected joint or backbone in a strip of at least 2-3 centimeter in length and at least 0.5 centimeter in diameter and massaged into the skin until completely absorbed, at least twice a day, and up to 5-6 times a day, wherein said formulation is used during at least three weeks, with the possibility of repeating the course if needed, thereby treatment is achieved. In a preferred embodiment, said method is a method of treatment of peripheral joints, spinal joints and/or extracellular matrix elements of connective tissue.

The proposed formulation for treatment of peripheral joints, spinal joints and/or extracellular matrix elements of connective tissue has a number of beneficial pharmaceutical properties. The major two of those are: 1) efficient restoration of articular cartilage and other extracellular matrix elements of connective tissue, comparable to that provided by injection of glucosamine, and 2) pronounced anesthetic effect comparable to, and sometimes even superior to, commercially available NSAID.

The beneficial pharmaceutical properties of the claimed formulation also include efficient prophylaxis of osteoarthritis and inflammatory diseases of joints and ligaments following traumas or related diseases after use of NSAID, stimulation of intra-articular fluid production and improvement of makeup thereof, as well as maintenance of optimal viscosity of intra-articular fluid.

The indicated beneficial pharmaceutical properties give rise to the following indications for use of the proposed formulation:

osteoarthritis, osteochondrosis of peripheral joints and spinal joints;

inflammatory diseases of joints, such as arthritis, including rheumatoid polyarthritis;

traumas of locomotor system elements, peripheral joints and spinal joints (bruises, ruptures, sprains);

prophylaxis of osteoarthrosis and inflammatory diseases of joints and ligaments following traumas;

protection of joints from side effects of NSAIDs and hormonal preparations on cartilaginous tissues (indometacin, orthofen, diclofenac, prednisolone);

muscle pains (mialgia, myositis).

### EXAMPLES

For the experimental studies, which are described in Examples 2-6 below, an exemplary formulation encompassed by the claimed invention, hereinafter referred to as Formulation A, has been prepared (see Example 1).

### Example 1. Preparation of Formulation A

The makeup of Formulation A is as follows:

| | |
|---|---|
| water | 71.69% by weight |
| glycerol | 3.0% by weight |
| butylene glycol | 2.0% by weight |
| cetyl alcohol | 2.3% by weight |
| glyceryl stearate | 3.8% by weight |
| caprylic/capric triglycerides | 5.0% by weight |
| cetyl octanoate | 1.0% by weight |
| dimethicone | 1.0% by weight |
| steareth-2 | 1.0% by weight |
| steareth-20 | 0.66% by weight |
| glucosamine sulfate potassium chloride | 8.00% by weight |
| methylisothiazolinone | 0.05% by weight |
| methylparaben | 0.2% by weight |
| peppermint oil | 0.3% by weight. |

To manufacture 100 kg of Formulation A, the following steps were taken.

(i) High-purity water in the amount of 71.69 kg is added to a first mixing tank, followed by addition of glycerol in the amount of 3.0 kg and butylene glycol in the amount of 2 kg; the mixture is heated to 70° C. with constant stirring at 60-70 rpm.

(ii) Then cetyl alcohol in the amount of 2.3 kg, glyceryl stearate in the amount of 3.8 kg, caprylic/capric triglycerides in the amount of 5.0 kg, cetyl octanoate in the amount of 1.0 kg, dimethicone in the amount of 1.0 kg, steareth-2 in the amount of 1.0 kg and steareth-20 in the amount of 0.66 kg are added to a second mixing tank; the mixture is heated to 70° C. with constant stirring at 60-70 rpm.

(iii) The mixture of (ii) is added to the first mixing tank with constant stirring at 60-70 rpm during 5 minutes, followed by cooling the mixture down to 50° C. or less and adding, with constant stirring at 60-70 rpm, glucosamine sulfate potassium chloride in the amount of 8.0 kg, followed by further cooling the mixture down to 30° C. and adding, with constant stirring at 60-70 rpm, methylisothiazolinone in the amount of 0.05 kg, methylparaben in the amount of 0.2 kg, and peppermint oil in the amount of 0.3 kg.

### Example 2. Structural Studies of Formulation A

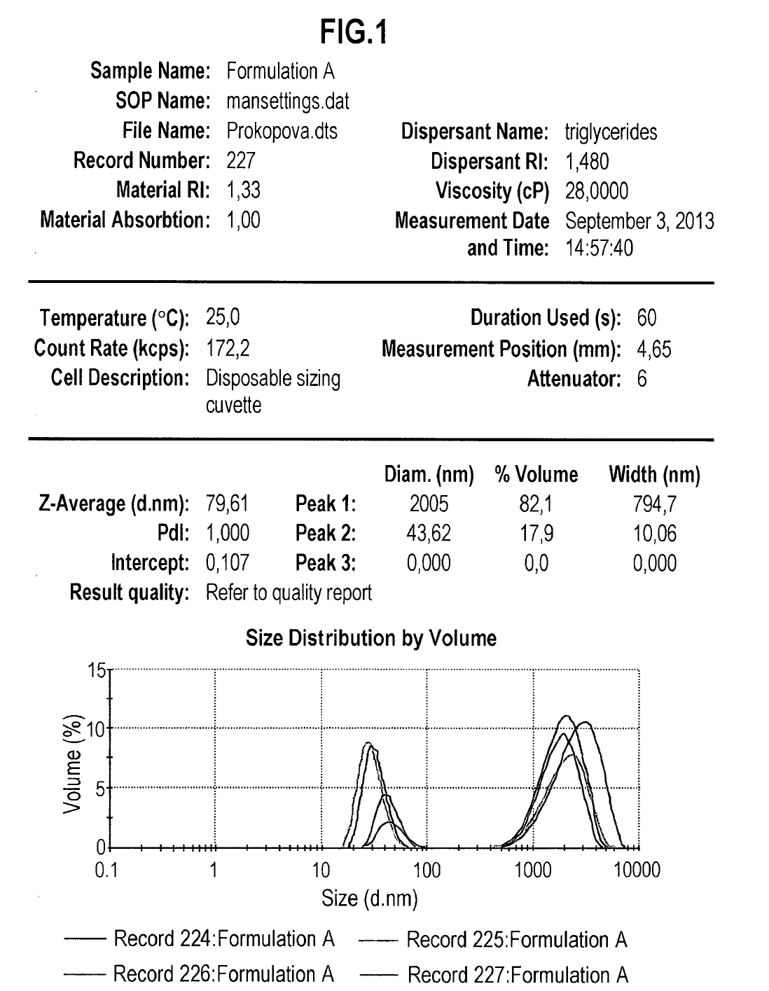

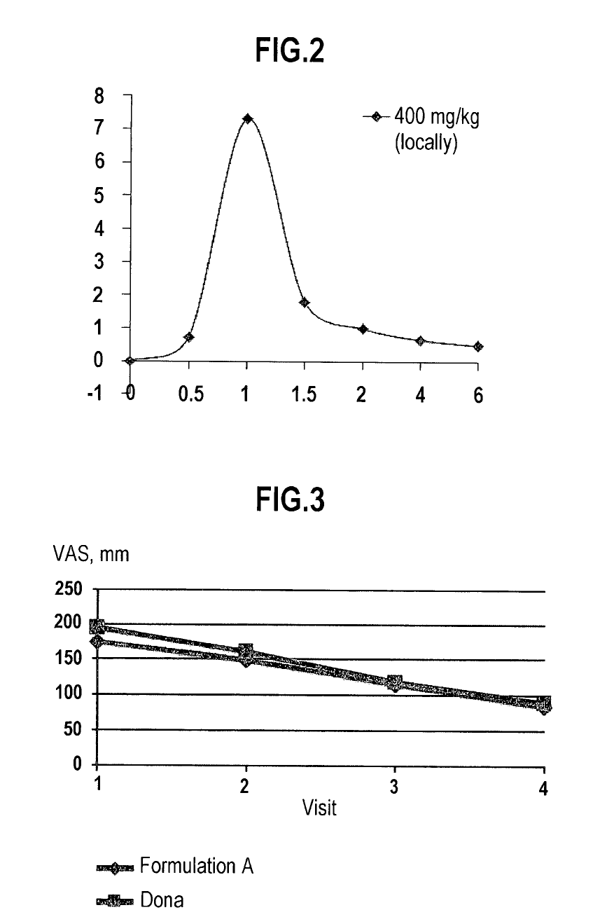

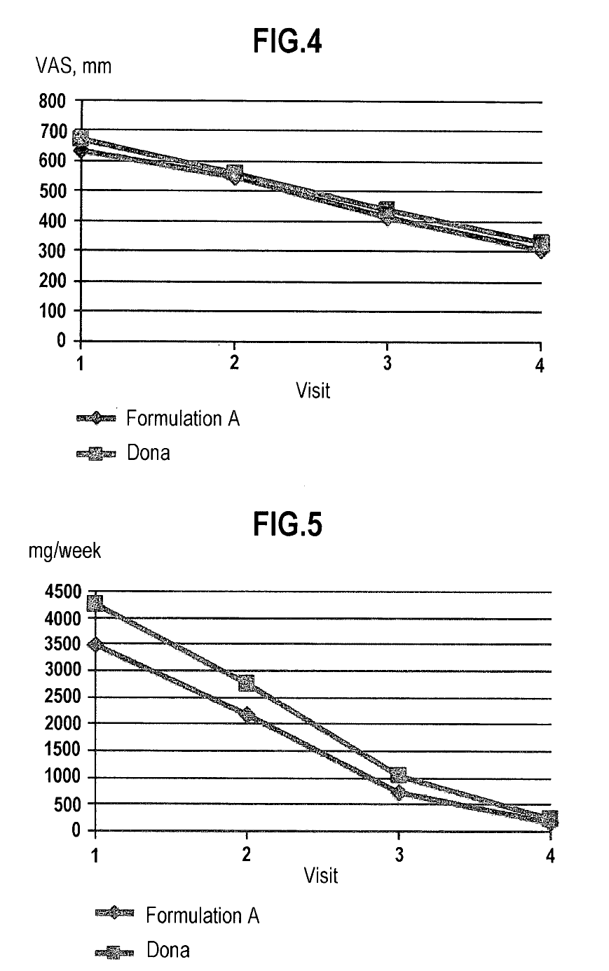

The aforementioned characteristics of the structures formed in the disperse system (hydrodynamic diameter/size of structures and their number by hydrodynamic diameter/size), have been determined by dynamic light scattering (photon correlation spectroscopy) using the Zetasizer Nano ZS device (Malvern, Great Britain) with laser radiation source at the wavelength of 532 nm (FIG. 1).

In FIG. 1, an automatically generated report shows the dependency of relative volume occupied by particles in the test sample on the particle size. For example, 17.9% of the total volume of the test sample occupied by particles corresponds to particles with a hydrodynamic diameter of 15 to 80 nm and the average hydrodynamic diameter is 43.62 nm, while 82.1% of the total volume of the test sample is occupied by particles corresponding to particles with a hydrodynamic diameter of 0.5 to 5 microns, and the average hydrodynamic diameter of this subset is 2005 nm.

### Example 3. Studies of Glucosamine Bioavailability in Use of Formulation A

For the purpose of obtaining objective information regarding glucosamine bioavailability following skin application, studies on rats have been carried out.

Studies comparing the relative bioavailability and penetration rate of glucosamine in cases of oral and intramuscular administration of aqueous glucosamine sulfate solutions and topical application of Formulation A cream have been conducted in Sprague-Dawley rats.

The results presented below have been obtained by determining blood plasma glucosamine concentrations in rats. Studies of 5 groups of rats, with 9 rats in each group were carried out in accordance with the main study protocol. One group was administered Formulation A cream topically, while another group was administered glucosamine solution orally and other three groups—via injections with varying glucosamine concentrations.

Based on analysis of pharmacokinetic profiles of glucosamine in blood plasma of rats, it has been established that the use of Formulation A cream providing a dose of 400 mg/kg of animal bodyweight 3 times a day for one week resulted in the bioavailability of 61.6% of glucosamine relative to intramuscular injection of 4% glucosamine sulfate solution containing a dose of 400 mg/kg 3 time a day for a one week time period. The average rate of glucosamine delivery through the skin of an animal into blood plasma over 4 hours following application of the cream was 26.9 μg/cm²·h. The relative amount of glucosamine delivered through the skin into blood plasma following a single application during a 4 hour time period was 4.12% of dose.

Comparative analysis of the experimental data obtained and those found in literature, as well as calculations based thereon, suggest that in the course of Formulation A treatment as administered in the experiment, the estimated average concentration of glucosamine in the synovial fluid of an inflamed joint is 0.7 to 1.5 μg/ml, which is 10-75 times higher than the endogenous glucosamine concentration in the synovial fluid of a human joint (typically 0.02 to 0.07 μgimp. Said value is comparable to the one achieved by administering glucosamine via injection, and is up to 2 times higher than the one achieved in case of oral forms of glucosamine.

### Example 4. Studies of Formulation A Stability

Stability of Formulation A was tested over time at three different conditions:
(i) incubation at 25±2° C. and relative humidity of 60±5%;
(ii) incubation at −15° C. followed by thawing;
(iii) incubation at 40±2° C. and relative humidity of 75±5%.
Formulation A was sealed in containers each containing 50 g of the drug. Three different containers were used for each of the three conditions. A number of physical and chemical parameters were monitored over a 3-year period of time at the following time points:

First year: every three months;

Second year: every six months;

Third year: once.

The results are presented below.

### Container No. 1 Date of Manufacture: 27 Mar. 2012

Conditions: temperature of 25±2° C. and relative humidity of 60±5%

| Test | 27 Mar. 2012 | 22 Jun. 2012 | 27 Sep. 2012 | 21 Mar. 2013 | 26 Sep. 2013 | 28 Mar. 2014 | 27 Mar. 2015 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.27 | 3.13 | 3.04 | 2.97 | 2.91 | 2.64 | 2.52 |
| Viscosity(#4.1.5 rpm) | 60000 | 96000 | 92000 | 80000 | 68000 | 55000 | 45000 |
| mPa · s | | | | | | | |
| 2500 G - force | No | NA | NA | NA | NA | NA | NA |
| Centrifuge | separation | | | | | | |
| Smell | Peppermint | conforms | conforms | conforms | conforms | conforms | conforms |
| Colour | P120C | P120C | P120C | P120C | P120C | P155C | P155C |
| Humidity | 60 | 60 | 60 | 61 | 61 | 61 | 62 |
| Temperature | 25 | 25.2 | 25.1 | 25 | 26 | 25.7 | 25.6 |

### Container No. 2 Date of Manufacture: 28 Mar. 2012

Conditions: temperature of 25±2° C. and relative humidity of 60±5%

| Test | 28 Mar. 2012 | 22 Jun. 2012 | 27 Sep. 2012 | 21 Mar. 2013 | 26 Sep. 2013 | 28 Mar. 2014 | 27 Mar. 2015 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.27 | 3.17 | 3.02 | 2.84 | 2.72 | 2.51 | 2.5 |
| Viscosity(#4.1.5 rpm) | 92000 | 104000 | 104000 | 92000 | 65000 | 52000 | 46000 |
| mPa · s | | | | | | | |
| 2500 G - Force | No | NA | NA | NA | NA | NA | NA |
| Centrifuge | separation | | | | | | |
| Smell | Peppermint | conforms | conforms | conforms | conforms | conforms | conforms |
| Colour | P120C | P120C | P120C | P120C | P120C | P155C | P155C |
| Humidity | 60 | 60 | 60 | 61 | 61 | 61 | 62 |
| Temperature | 25 | 25.2 | 25.1 | 25 | 26 | 25.7 | 25.6 |

### Container No. 3 Date of Manufacture: 28 Mar. 2012

Conditions: temperature of 25±2° C. and relative humidity of 60±5%

| Test | 28 Mar. 2012 | 22 Jun. 2012 | 27 Sep. 2012 | 21 Mar. 2013 | 26 Sep. 2013 | 28 Mar. 2014 | 27 Mar. 2015 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.31 | 3.13 | 3.06 | 2.88 | 2.72 | 2.64 | 2.51 |
| Viscosity(#4.1.5 rpm) | 96000 | 102000 | 98000 | 88000 | 68000 | 56000 | 46000 |
| mPa · s | | | | | | | |
| 2500 G - Force | No | NA | NA | NA | NA | NA | NA |
| Centrifuge | separation | | | | | | |
| Smell | Peppermint | conforms | conforms | conforms | conforms | conforms | conforms |
| Colour | P120C | P120C | P120C | P120C | P120C | P155C | P155C |
| Humidity | 60 | 60 | 60 | 61 | 61 | 61 | 62 |
| Temperature | 25 | 25.2 | 25.1 | 25 | 26 | 25.7 | 25.6 |

### Container No. 4 Date of Manufacture: 27 Mar. 2012

Conditions: incubation at −15° C. followed by thawing

| Test | 27 Mar. 2012 | 22 Jun. 2012 | 27 Sep. 2012 | 21 Mar. 2013 | 26 Sep. 2013 | 28 Mar. 2014 | 27 Mar. 2015 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.27 | 4.32 | 4.02 | 3.87 | 3.65 | 3.54 | 3.38 |
| Viscosity(#4.1.5 rpm) | 60000 | 55000 | 55000 | 52000 | 52000 | 52000 | 52000 |
| mPa · s | | | | | | | |
| 2500 G - Force | No | NA | NA | NA | NA | NA | NA |
| Centrifuge | separation | | | | | | |
| Smell | Peppermint | conforms | conforms | conforms | conforms | conforms | conforms |
| Colour | P120C | P120C | P120C | P120C | P120C | P120C | P120C |
| Temperature | 25 | 25.2 | 25.1 | 25 | 26 | 25.7 | 25.6 |

### Container No. 5 Date of Manufacture: 28 Mar. 2012

Conditions: incubation at −15° C. followed by thawing

| Test | 28 Mar. 2012 | 22 Jun. 2012 | 27 Sep. 2012 | 21 Mar. 2013 | 26 Sep. 2013 | 28 Mar. 2014 | 27 Mar. 2015 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.27 | 4.32 | 4.02 | 3.91 | 3.82 | 3.62 | 3.45 |
| Viscosity(#4.1.5 rpm) | 92000 | 84000 | 80000 | 68000 | 54000 | 52000 | 52000 |
| mPa · s | | | | | | | |
| 2500 G - Force | No | NA | NA | NA | NA | NA | NA |
| Centrifuge | separation | | | | | | |
| Smell | Peppermint | conforms | conforms | conforms | conforms | conforms | conforms |
| Colour | P120C | P120C | P120C | P120C | P120C | P120C | P120C |
| Temperature | 25 | 25.2 | 25.1 | 25 | 26 | 25.7 | 25.6 |

### Container No. 6 Date of Manufacture: 28 Mar. 2012

Conditions: incubation at −15° C. followed by thawing

| Test | 28 Mar. 2012 | 22 Jun. 2012 | 27 Sep. 2012 | 21 Mar. 2013 | 26 Sep. 2013 | 28 Mar. 2014 | 27 Mar. 2015 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.31 | 4.47 | 4.02 | 3.91 | 3.82 | 3.63 | 3.44 |
| Viscosity(#4.1.5 rpm) | 96000 | 86000 | 82000 | 68000 | 54000 | 52000 | 52000 |
| mPa · s | | | | | | | |
| 2500 G - Force | No | NA | NA | NA | NA | NA | NA |
| Centrifuge | separation | | | | | | |
| Smell | Peppermint | conforms | conforms | conforms | conforms | conforms | conforms |
| Colour | P120C | P120C | P120C | P120C | P120C | P120C | P120C |
| Temperature | 25 | 25.2 | 25.1 | 25 | 26 | 25.7 | 25.6 |

### Container No. 7 Date of Manufacture: 27 Mar. 2012

Conditions: incubation at 40±2° C. and relative humidity of 75±5%

| Test | 30 Dec. 1999 | 3 Apr. 2012 | 3 May 2012 | 27 Jun. 2012 | 3 Jul. 2012 | 3 Aug. 2012 | 27 Sep. 2012 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.27 | 3.58 | 3.14 | 3.14 | 2.97 | 2.41 | 2.38 |
| Viscosity(#4.1.5 rpm) | 60000 | 58000 | 54000 | 50000 | 48000 | 44000 | 42000 |
| mPa · s | | | | | | | |
| G - Force | no | conforms | conforms | conforms | conforms | conforms | conforms |
| | separation | | | | | | |
| Smell | Peppermint | conforms | conforms | conforms | conforms | Fruity | Fruity |
| Colour | P120C | P120C | P120C | P155C | P155C | P155C | P155C |
| Humidity | 75 | 75 | 76 | 76 | 76 | 75 | 76 |
| Temperature | 25 | 25.1 | 25 | 25 | 25.2 | 26 | 25.6 |

### Container No. 8 Date of Manufacture: 28 Mar. 2012

Conditions: incubation at 40±2° C. and relative humidity of 75±5%

| Test | 28 Mar. 2012 | 3 Apr. 2012 | 3 May 2012 | 27 Jun. 2012 | 3 Jul. 2012 | 3 Aug. 2012 | 27 Sep. 2012 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.27 | 3.54 | 3.21 | 3.06 | 2.93 | 2.44 | 2.42 |
| Viscosity(#4.1.5 rpm) | 92000 | 88000 | 84000 | 82000 | 78000 | 66000 | 46000 |
| mPa · s | | | | | | | |
| G - Force | No | conforms | conforms | conforms | conforms | conforms | conforms |
| | separation | | | | | | |
| Colour | P120C | P120C | P120C | P155C | P155C | P155C | P155C |
| Humidity | 75 | 75 | 76 | 76 | 76 | 75 | 76 |
| Temperature | 25 | 25.1 | 25 | 25 | 25.2 | 26 | 25.6 |

### Container No. 9 Date of Manufacture: 28 Mar. 2012

Conditions: incubation at 40±2° C. and relative humidity of 75±5%

| Test | 28 Mar. 2012 | 3 Apr. 2012 | 3 May 2012 | 28 Jun. 2012 | 3 Jul. 2012 | 3 Aug. 2012 | 27 Sep. 2012 |
|---|---|---|---|---|---|---|---|
| Physical Appearance | conforms | conforms | conforms | conforms | conforms | conforms | conforms |
| (initially: shiny and | | | | | | | |
| homogeneous cream) | | | | | | | |
| pH | 5.31 | 3.61 | 3.18 | 3.02 | 2.95 | 2.48 | 2.42 |
| Viscosity(#4.1.5 rpm) | 96000 | 90000 | 86000 | 84000 | 80000 | 68000 | 48000 |
| mPa · s | | | | | | | |
| G - Force | No | conforms | conforms | conforms | conforms | conforms | conforms |
| | separation | | | | | | |
| Smell | peppermint | conforms | conforms | conforms | conforms | Fruity | Fruity |
| Colour | P120C | P120C | P120C | P155C | P155C | P155C | P155C |
| Humidity | 75 | 75 | 76 | 76 | 76 | 75 | 76 |

The results indicate that despite the decrease in pH and viscosity, the product remains a homogeneous cream and is efficacious for at least three years.

### Example 5. Comparison of Formulation A and Voltaren® Emulgel®

In order to confirm the efficacy of the claimed formulation, comparative randomized clinical trials have been carried out to test the efficiency, safety and tolerability of Formulation A versus those of Voltaren® Emulgel® (gel for external use, NOVARTIS Consumer Health GmbH, Switzerland) in 80 volunteers diagnosed with stage I-III osteoarthrosis of the knee joint.

Of the 80 patients, 68 were women and 12 were men between the age of 44 and 80. As a result of screening and randomization, all of the patients complied with the inclusion criteria.

The patients were randomized into two groups: the experimental group (Formulation A) and the control group (Voltaren® Emulgel®). All the patients completed the study according to the protocol. The two groups did not statistically differ in terms of age and sex, height and weight, systolic and diastolic blood pressure, articular syndrome clinical score (the Western Ontario and McMaster Universities Arthritis Index, or “WOMAC”, used for pain assessment in the target joint during walking and on palpation using visual analog scale (VAS)). No statistically significant difference in terms of radiological stages of gonarthrosis between the two groups was established. No reliable differences between the two groups in most laboratory indicators were revealed either. Based on the aforesaid, the two groups may be considered comparable.

No clinically significant deviations in blood pressure, pulse, electrocardiogram, or laboratory indicators were observed in the course of the studies. Throughout the study, 11 mild adverse events were detected. Two adverse events (one in each group) associated with the administered medicament were detected. Both were mild, did not require treatment and ended in recovery. The primary endpoint, which corresponds to “reduction by more than 20%” in “WOMAC Pain”, was reached by 34 and 30 patients in the experimental and control groups, respectively. Further, the effect persisted after withdrawal of the treatment: at visit 4 (on 56 day from start of therapy), 32 and 24 patients, respectively, still met the endpoint. Comparison of the results between the two groups did not reveal significant differences between the experimental and the control groups. Therefore, in terms of the above-mentioned criterion, the efficiency of treatment by Formulation A did not differ statistically from the efficiency of treatment by Voltaren® Emulgel® (as concluded by the physicians conducting the study).

Four weeks following the end of the treatment, the number of positive results was statistically significant in the experimetal group, according to both patients and clinicians. In other words, the transdermal delivery system for glucosamine was found to be equivalent to Voltaren® Emulgel® for reduction of pain and superior to Voltaren® Emulgel® in terms of duration of therapeutic effect.

### Example 6. Comparison of Formulation a with a Glucosamine Sulfate Injection Form, “Dona”

To further confirm the efficacy of the transdermal delivery system for glucosamine of the invention, the inventors of the present invention have conducted comparative studies of therapeutic efficiency of Formulation A (cream for external use) and an injection form of glucosamine sulfate for intramuscular administration “Dona” (ampoules for injection 2 ml each, contains 200 mg of glucosamine sulfate potassium chloride in 1 ml) in patients diagnosed with gonarthrosis in treating osteoarthritis of knee joint.

Prior to the studies, 40 patients having confirmed osteoarthritis of knee joint (gonarthrosis) (in accordance with R. Altman criteria, 1995) and having pronounced pain syndrome were evaluated. The patients aged from 48 to 75 (average age 56.7±8.2 years); 38 (95%) were women and 2 (5%) men. The duration of the disease varied from 2 to 15 years (average duration 6.7±2.9 years). In accordance with the Kellegren & Lawrence classification, in 30 (75%) patients the radiographic grade of gonarthrosis was determined to be II and in 10 (25%) patients it was determined to be III. At the time of the evaluation, all the 40 (100%) patients were receiving NSAID treatment.

The patients were divided into two groups (20 people each) comparable in terms of main parameters. The first group was receiving Formulation A topically, wherein strip 2-3 centimeters length and about 0.5 centimeters in diameter of the cream was being applied to the affected joint area three times a day for one month. The joint targeted by the treatment was the one that had been giving most pain at the start of the study. The second group of patients was receiving “Dona” intramuscularly once a day, three times a week during one month. The efficiency of the therapy was evaluated in accordance with the dynamics of articular syndrome indices in the target knee joint. The following factors were evaluated: pain intensity in the joint at rest and in motion using VAS, dynamics of a total index and of separate indices: pain, constraint and functional insufficiency of a joint by the WOMAC score, need for NSAID (ibuprofen). The above-mentioned clinical parameters were assessed at the first evaluation and once a week during one month.

As a result of the aforementioned therapy, most dynamics in comparing WOMAC indices has been observed in case of the index characterizing reduction in severity of pain syndrome. Over the course of the treatment, significant (p<0.05) reduction in pain associated with walking on flat surface, going up the stairs, palpation of joints was observed (see Table 1, FIG. 1, 2). No statistically significant difference in the indicators between the two groups was observed. However, more pronounced (p=0.06) reduction in painful sensation caused by palpation was observed in case of treatment with Formulation A, which may be explained perhaps by its topical application directly to the painful sites.

In both groups, a reliably significant (p<0.05) improvement of physical activity was observed, which was manifested in the reduction of the time the patient required to walk a 15 meter distance from 29.6±12.18 s to 18.0±6.1 s and from 30.7±12.21 s to 18.25±5.23 s in the first and second group, respectively (see Table 1).

In general, the reduction in the total WOMAC index observed in Formulation A patients towards the end of the study (57%) did not differ statistically from that observed in the reference “Dona” group (more than 56%) (see FIG. 3, 4)

Over the course of treatment each group showed clear reduction of average need for NSAID by more than 15 times (see Table 1, FIG. 5). In more detail, of the patients receiving Formulation A, 15 (75%) stopped receiving ibuprofen, while 5 (25%) had the dose reduced 7-10 fold. In the group receiving “Dona”, after 1 month of the treatment 16 (80%) patients no longer needed ibuprofen, whereas 4 patients had the original dose reduced by 3-5 times.

TABLE 1
Articulate syndrome quantitative indices in cases of Formulation
A (topically) and “Dona” (intramuscularly)

| | Formulation A Group (n = 20) | | “Dona” Group (n = 20) | |
|---|---|---|---|---|
| | Before | After | Before | After |
| Indicators | treatment | treatment | treatment | treatment |
| Pain on palpation, | 52.54 ± 9.87 | 23.69 ± 10.2* ** | 51.26 ± 9.19 | 31.94 ± 13.91* |
| VAS mm | | | | |
| Pain when | 52.25 ± 12.7 | 29.75 ± 16.23* | 55 ± 9.8* | 27.25 ± 12.18* |
| walking, VAS mm | | | | |
| Pain when going | 60.3 ± 12.31 | 29.6 ± 15.38* | 64.45 ± 8.505 | 33.45 ± 13.48* |
| up the stairs, VAS mm | | | | |
| WOMAC, total | 829 ± 191 | 404.3 ± 274.15* | 893.5 ± 220.7 | 433.1 ± 282.16* |
| index, points | | | | |
| Time taken by | 29.6 ± 12.18 | 18.6 ± 6.1* | 30.7 ± 12.21 | 18.25 ± 5.225* |
| walking 15 m, sec | | | | |
| Weekly NSAID | 3480 ± 1200 | 180 ± 255* | 4250 ± 1325 | 240 ± 384* |
| need (ibuprofen), mg | | | | |

*P < 0.05 - significant difference between indicators in every group before and after the treatment;
** P = 0.06 - significant difference between indicators in the groups after administering Formulation A topically and “Dona” intramuscularly.

The main conclusion from the studies described herein is that the proposed formulation is similar and, in some cases superior to, a commercially available glucosamine sulfate-based preparations for injections, both in terms of anesthetic effect and restoration of mobility of affected joint, as well as reduction of average weekly need for NSAID in the course of treatment.

### THE LITERATURE CITED

1. Altman, R. D. Clinical Pharmacology 2(4), 359-371 (2009) [in Russian].
2. Handbook on Drugs, Vidal: http://www.vidal.ru/drugs/molecule/322.
3. Scartazzini R., Luisi P. L., Organogels from Lecithins//J. Phys. Chem. 92, 829-833 (1988).

1. A formulation comprising one or more pharmaceutically active agents in an effective amount and at least the following ingredients:
60% to 80% by weight of water,
1.0% to 6.0% by weight of glycerol,
0.7% to 4% by weight of butylene glycol,
up to 12.1% by weight of C8 fatty alcohols,
up to 3.5% by weight of C16 fatty alcohols,
up to 4.2% by weight of C21 fatty alcohols,
up to 2.0% by weight of esters of C15-C17 fatty acids,
up to 1.4% by weight of polysiloxane,
up to 1.4% by weight of C2 polyglycol,
up to 0.98% by weight of C20 polyglycol.

2. (canceled)

3. The formulation according to claim 1, wherein ingredients in said formulation form an aggregative and sedimentation stable disperse system, which comprises micellar and/or liposomal structures.

4. The formulation according to claim 3, wherein said structures form at least one group, in which the hydrodynamic diameter/size of at least 90% of the structures is between 15 and 100 nm, and the maximum of the distribution of the number of particles by size is between 25 and 50 nm.

5. The formulation according to claim 3, wherein said structures form at least two groups, wherein
in the first group the hydrodynamic diameter/size of at least 90% of the structures is between 15 and 100 nm, and the maximum of the distribution of the number of particles by size is between 25 and 50 nm, and
in the second group the hydrodynamic diameter/size of at least 90% of the structures is between 500 and 7000 nm, and the maximum of the distribution of the number of particles by size is between 1500 and 5000 nm.

6. The formulation according to claim 1, wherein said one or more pharmaceutically active ingredients is a glucosamine salt.

7. The formulation according to claim 6, wherein the glucosamine salt is glucosamine sulfate.

8. The formulation according to claim 6, wherein the glucosamine salt is glucosamine sulfate potassium chloride.

9. The formulation according to claim 8, wherein glucosamine sulfate potassium chloride is present in the formulation in the amount of 4 to 14% by weight.

10. The formulation according to claim 1, further comprising amino polycarboxylic acid in the amount of 0.05-0.3% by weight.

11. The formulation according to claim 1, further comprising hydroxyethyl cellulose in the amount of 0.12-1.0% by weight.

12. The formulation according to claim 1, further comprising dimethyl sulfide (DMS) in the amount of 0.5-5% by weight.

13. The formulation according to claim 1, further comprising ascorbic acid in the amount of 0.05-0.3% by weight.

14. The formulation according to claim 1, further comprising capsaicinoid, in particular, capsaicin, in the amount of 0.05-0.5% by weight.

15. The formulation according to claim 1, further comprising camphor in the amount of 0.2-1.8% by weight.

16. The formulation according to claim 1, further comprising silicagel in the amount of 0.005-0.05% by weight.

17. The formulation according to claim 1, further comprising ginger extract in the amount of 0.00005-0.005% by weight.

18. The formulation according to claim 1, wherein said formulation further comprises methylparaben in the amount of 0.01-0.3% by weight and/or methylisotiazolinone in the amount of 0.01-0.05% by weight.

19. The formulation according to claim 1, wherein said formulation further comprises a mixture of salix alba bark extract with propanediol (ISCAGUARD® SAP) in the amount of 0.02-2.5% by weight.

20. The formulation according to claim 1, wherein said formulation further comprises perfume, peppermint oil and/or menthol in the amount of 0.02-1.5% by weight.

21. The formulation according to claim 1, further comprising silicagel in the amount of 0.005-0.05% by weight.

22. The formulation according to claim 1, further comprising Tris(hydroxymethyl)aminomethane (HOCH₂)3CNH₂ (Tris) in the amount of 0.1-0.9% by weight.

23. A formulation according to claim 1, comprising:

| | |
|---|---|
| water | 71.69% by weight |
| glycerol | 3.0% by weight |
| butylene glycol | 2.0% by weight |
| cetyl alcohol | 2.3% by weight |
| glyceryl stearate | 3.8% by weight |
| caprylic/capric triglycerides | 5.0% by weight |
| cetyl octanoate | 1.0% by weight |
| dimethicone | 1.0% by weight |
| steareth-2 | 1.0% by weight |
| steareth-20 | 0.66% by weight |
| glucosamine sulfate potassium chloride | 8.00% by weight |
| methylisothiazolinone | 0.05% by weight |
| methylparaben | 0.2% by weight |
| peppermint oil | 0.3% by weight. |

24. A method of producing a formulation for the treatment of peripheral joints, spinal joints and/or extracellular matrix elements of connective tissue, comprising:
(i) Adding high-purity water in the amount of 60-80% by weight to a first mixing tank adding glycerol in the amount of 1-6% by weight; adding butylene glycol in the amount of 0.7-4% by weight; heating the mixture to 65-70° C. with constant stirring;
(ii) Adding to a second mixing tank: one or more C16 fatty alcohols in the amount of 0.3-3.5% by weight, one or more C21 fatty alcohols in the amount of 1-4.2% by weight, one or more C8 fatty alcohols in the amount of 1.7-12.1% by weight, one or more esters of C15-C17 fatty acids in the amount of 0.3-2.0% by weight, one or more polysiloxanes in the amount of 0.3-1.4% by weight, one or more C2 polyglycols in the amount of 0.3-1.4% by weight, and one or more C20 polyglycols in the amount of 0.22-0.98% by weight; and heating the mixture to 65-70° C. with constant stirring;
(iii) Adding the mixture of (ii) to the first mixing tank with constant stirring during a 5 minute period; cooling the mixture down to 50° C. or less; adding, with constant stirring, one or more pharmaceutically active agents; cooling the mixture to 30° C.; and adding, with constant stirring, preservative(s) and perfume,
thereby producing a formulation for treating of peripheral joints, spinal joints and/or extracellular matrix elements of connective tissue.

25. The method according to claim 24, wherein said one or more pharmaceutically active agents is a glucosamine salt.

26. The method according to claim 25, wherein the glucosamine salt is glucosamine sulfate.

27. The method according to claim 25, wherein the glucosamine salt is glucosamine sulfate potassium chloride.

28. The method according to claim 24, further comprising at step (i) adding amino polycarboxylic acid in the amount of 0.05-0.3% by weight to the first mixing tank before the heating.

29. The method according to claim 24, further comprising at step (i) adding hydroxyethyl cellulose in the amount of 0.12-1.0% by weight to the first mixing tank before the heating.

30. The method according to claim 24, further comprising, at step (iii) adding DMS in the amount of 0.5-5% by weight after the addition of one or more pharmaceutically active agents.

31. The method according to claim 24, further comprising, at step (iii) adding ascorbic acid in the amount of 0.05-0.3% by weight after the addition of one or more pharmaceutically active agents.

32. The method according to claim 24, wherein at step (ii) capsaicin, in the amount of 0.05-0.5% by weight is added to the second mixing tank after the temperature of 65° C. is reached.

33. The method according to claim 24, wherein at step (i) silicagel in the amount of 0.005-0.05% by weight is added to the first mixing tank before heating.

34. The method according to claim 24, wherein at step (ii) ginger extract in the amount of 0.00005-0.005% by weight is added to the second mixing tank after the cooling down to 50° C.

35. The method according to claim 24, wherein at step (iii) methylparaben in the amount of 0.01-0.3% by weight and/or methylisotiazolinone in the amount of 0.01-0.05% by weight is added.

36. The method according to claim 24, wherein at step (iii) a mixture of salix alba bark extract with propanediol (ISCAGUARD® SAP) in the amount of 0.02-2.5% by weight is added.

37. The method according to claim 24, wherein at step (iii) peppermint oil and/or menthol in the amount of 0.02-1.5% by weight is added.

38. The method according to claim 24, wherein at step (i) Tris (hydroxymethyl) aminomethane in the amount of 0.1-0.9% by weight is added after the addition of water to the first mixing tank.

39. A formulation produced by the method according to claim 24.

40. A method of treatment, the method comprising applying the formulation according to claim 1 onto the skin of a patient at the site of an affected joint or backbone in a strip of at least 2-3 centimeter in length and at least 0.5 centimeter in width and massaged into the skin until completely absorbed, at least twice a day and up to 5-6 times a day, wherein said formulation is used daily for a period of at least three weeks and repeating until treatment is achieved.

41. The method according to claim 40, wherein said method of treatment is a method of treatment for peripheral joints, spinal joints and/or extracellular matrix elements of connective tissue.
